(19) United States
(12) Patent Application Publication
(10) Pub. No.: US 2023/0256191 A1
(43) Pub. Date: Aug. 17, 2023

(54) NON-AUDITORY NEUROSTIMULATION AND METHODS FOR ANESTHESIA RECOVERY

(71) Applicant: BrainFM, Inc., Brooklyn, NY (US)

(72) Inventor: Kevin J.P. Woods, Brooklyn, NY (US)

(73) Assignee: BrainFM, Inc., Brooklyn, NY (US)

(21) Appl. No.: 17/804,407

(22) Filed: May 27, 2022

Related U.S. Application Data
(60) Provisional application No. 63/268,168, filed on Feb. 17, 2022.

(51) Int. Cl. A61M 21/00

(52) U.S. Cl. CPC A61M 21/00; A61M 2021/0083; A61M 2021/0027; A61M 2205/3303; A61M 2205/50

(57) ABSTRACT

Techniques (methods and devices) for neural stimulation through audio and/or non-audio stimulation. The techniques may be performed by a processing device and may include receiving an audio signal from an audio source and a desired mental state. An element of the audio signal that correspond to a modulation characteristic of the desired mental state may be identified. An envelope from the element may be determined. One or more non-audio signals may be generated based on at least a rate and phase of the envelope. The one or more non-audio signals may be transmitted to one or more non-audio output devices to generate one or more non-audio outputs. A relative timing of the one or more non-audio outputs and an output of the audio signal may be coordinated. The neural stimulation through audio and/or non-audio stimulation may assist patients before, during, and after anesthesia.

### CROSS-REFERENCE TO RELATED APPLICATIONS

This application claims benefit to U.S. Patent App. No. 63/268,168 entitled “Perioperative Functional Audio for Anxiety and Cognitive Recovery From Anesthesia” and filed on Feb. 17, 2022 and is related to U.S. patent application Ser. No. 17/366,896 entitled “Neural Stimulation Through Audio with Dynamic Modulation Characteristics” and filed on Jul. 2, 2021, U.S. patent application Ser. No. 17/505,453 entitled “Audio Content Serving and Creation Based on Modulation Characteristics” and filed on Oct. 18, 2021, and U.S. patent application Ser. No. 17/556,583 entitled “Extending Audio Tracks While Avoiding Audio Discontinuities” and filed on Dec. 20, 2021, the entire contents of which are incorporated herein by reference.

### TECHNICAL FIELD

The present disclosure relates to neural stimulation, particularly, noninvasive neural stimulation using one or more of auditory and non-auditory sensory modalities such that multi-modal entrainment may be used to increase the benefit of neurological stimulation. Additionally, this disclosure also captures a novel use of sensory neuromodulation for recovery from anesthesia.

### BACKGROUND

For decades, neuroscientists have observed wave-like activity in the brain called neural oscillations. Various aspects of these oscillations have been related to mental states including alertness, attention, relaxation, and sleep. The ability to effectively induce and modify such mental states by noninvasive brain stimulation through one or more modalities (e.g., audio and non-audio) is desirable.

### SUMMARY

Techniques for neural stimulation through audio and/or non-audio stimulation are disclosed. The techniques may be performed by a processing device and may include receiving an audio signal from an audio source and receiving a desired mental state for a user. An element of the audio signal that contains modulation characteristics corresponding to the desired mental state may be identified. An acoustic envelope of the element may be determined. One or more signals may be generated based on at least a rate and phase of the envelope. The one or more signals may be transmitted to one or more non-audio output devices to generate one or more non-audio outputs. The non-audio outputs may occur concurrently with audio outputs. A relative timing of the one or more non-audio outputs and an output of the audio signal may be coordinated using one or more of predetermined models and/or sensor data.

The neural stimulation of a patient through audio and/or non-audio stimulation may assist the patient before, during, and after anesthesia is administered to the patient. One method may include administering rhythmic stimulation having a sedative effect prior to administration of the anesthesia to the patient. Another method may include administering rhythmic stimulation having a stimulative effect after administration of the anesthesia has concluded. The rhythmic stimulation may include (i) one or more audio outputs generated by one or more audio playback devices that minimize the audio's impact on a patient's situational awareness and provides audible sound only to the patient via a limited sound field or headphones, and/or (ii) one or more non-audio outputs generated by non-audio stimulation devices. The one or more audio playback devices may include, for example, one or more of bone-conduction headphones, audio headphones, and audio speakers (e.g., passive speakers, smart speakers, etc.). The one or more non-audio stimulation devices may include, for example, one or more wearables, a connected vibrating bed, an electrical brain-stimulation device, and one or more lights. The modifying may occur while the patient is conscious or unconscious, and may be performed by one or more of a manual selection by a caregiver or an automatic selection based on one or more sensors. One or more characteristics of the rhythmic stimulation may be adjusted via (i) manual input by the patient and/or a caregiver, and/or (ii) automatic input based on one or more sensors. The one or more characteristics may include, for example, gain and modulation depth.

### BRIEF DESCRIPTION OF DRAWINGS

Other objects and advantages of the present disclosure will become apparent to those skilled in the art upon reading the following detailed description of exemplary embodiments and appended claims, in conjunction with the accompanying drawings, in which like reference numerals have been used to designate like elements, and in which:

FIG. 1 depicts a flow diagram of an illustrative method for coordinating modulation in multiple input modalities to the central nervous system, according to an exemplary embodiment;

FIG. 2 depicts a flow diagram illustrating details of an audio analysis, according to an exemplary embodiment;

FIG. 3 depicts a flow diagram illustrating details of a generation of non-audio stimulus, according to an exemplary embodiment;

FIG. 4 depicts a flow diagram illustrating details of using sensor data to determine effects of multimodal stimulation, according to an exemplary embodiment;

FIG. 5 depicts a functional block diagram of an example processing device, according to an exemplary embodiment;

FIG. 6 depicts a functional block diagram that illustrates an example system, according to an exemplary embodiment;

FIG. 7 depicts a flow diagram of an illustrative method for using rhythmic stimulation to improve patient satisfaction and performance before, during, and after anesthesia, according to an exemplary embodiment;

FIG. 8A depicts a plot showing a patient's willingness to recommend audio they received to aid recovery during the emergence from anesthesia to family and friends if undergoing the same procedure, according to an exemplary embodiment; and

FIG. 8B depicts a plot showing an average time to discharge a patient once the patient is in recovery, according to an exemplary embodiment.

The figures are for purposes of illustrating example embodiments, but it is understood that the inventions are not limited to the arrangements and instrumentality shown in the drawings. In the figures, identical reference numbers identify at least generally similar elements.

### DETAILED DESCRIPTION

I. Overview

The present disclosure describes systems, methods, apparatuses and non-transitory computer executable media configured to generate multimodal stimulation (e.g., with multiple input channels to the body and/or the brain) targeted to affect a desired mental state for a user. As described below, models and/or sensor data may be used to guide stimulation parameters and to find audio features conducive to producing a desired mental state, and transferring such features to either a stimulus in another sensory modality (e.g., touch/vibration, light/vision, taste/chemoreception, smell/olfaction, temperature), or a stimulating signal (electrical or magnetic stimulation).

Non-audio modulation may be created to enforce audio modulation at a particular rate (e.g., to target a particular mental state), even if the audio contains modulation energy at many rates. The relative phase (timing/delay) of modulation across the modalities may be a factor. The combined effect on the brain of the multimodal stimulation (e.g., auditory and non-auditory) may be adjusted by changing aspects of the non-audio modulation, such as phase (i.e., relative to the audio modulation), waveform shape, rate and/or depth. This may increase the entrainment due to multimodal stimulation if desired.

In various examples described herein, neurostimulation may be delivered by a non-audio signal in combination with an audio signal. According to such examples, the non-audio signal may be based on the audio signal such that both the non-audio signal and the audio signal produce the same desired mental state. The non-audio signal may affect the brain differently than the audio signal, and delivery of both the non-audio and audio signals concurrently may affect the brain differently than would delivery of either signal alone. The combination of signals may be more effective than either signal alone at producing or sustaining a mental state in the user.

Further, a use of audio and/or non-audio neurostimulation for recovery from anesthesia is described herein. In particular, a procedure is described that outlines a process for using audio and/or non-audio stimulation to initiate cognition after anesthesia is administered. This stimulation may be delivered, for example, through audio using traditional headphones/speakers, non-auditory sensory modalities (e.g., light, touch), and/or non-sensory neural stimulation (e.g., transcranial direct-current stimulation).

Modulation characteristics of signals may include depth of modulation at a certain rate, the rate itself, modulation depth across all rates (i.e., the modulation spectrum), phase at a rate, among others. These modulation characteristics may be from a broadband portion of a signal or in sub-bands (e.g., frequency regions, such as bass vs. treble) of the signal. Audio/audio element, as used herein, may refer to a single audio element (e.g., a single digital file), an audio feed (either analog or digital) from a received signal, or a live recording. Modulation characteristics may exist in a non-audio signal, for example the output of a flashing light may be described in terms of modulation rate, depth, phase, waveshape, and other modulation characteristics. Fluctuations in intensity over time of sensory (sound, light) and non-sensory (electrical current, magnetic field strength) signals can be quantified in this way.

In various exemplary embodiments described herein, the presently disclosed techniques may be effective to affect a desired mental state when audio stimulation is provided at predetermined frequencies, which are associated with known portions of the cochlea of the human ear and may be referenced in terms of the cochlea, or in terms of absolute frequency. Furthermore, the presently disclosed techniques may provide for a selection of modulation characteristics configured to target different patterns of brain activity. These aspects are subsequently described in detail.

In various exemplary embodiments described herein, audio and/or non-audio stimulation may be generated to change over time according to a stimulation protocol to affect patterns of neural activity in the brain to affect mental state, behavior, and/or mood. Modulation may be added to audio (e.g., mixed) which may in turn be stored and retrieved for playback at a later time. Modulation may be added (e.g., mixed) to audio for immediate (e.g., real-time) playback. Modulated audio playback may be facilitated from a playback device (e.g., smart speaker, headphone, portable device, computer, etc.) and may be single or multi-channel audio. Users may facilitate the playback of the modulated audio through, for example, an interface on a processing device (e.g., smartphone, computer, etc.).

In various examples described herein, audio may also be analyzed, and this analysis may be used to generate non-audio stimulation which may be delivered by one or more non-audio stimulation devices. These aspects are subsequently described in more detail below.

The present disclosure will now be described more fully hereinafter with reference to the accompanying drawings, which form a part hereof, and which show, by way of non-limiting illustration, certain examples. Subject matter may, however, be described in a variety of different forms and, therefore, covered or claimed subject matter is intended to be construed as not being limited to any examples set forth herein. Among other things, subject matter may be described as methods, devices, components, or systems. Accordingly, examples may take the form of hardware, software, firmware or any combination thereof (other than software per se). The following detailed description is, therefore, not intended to be taken in a limiting sense.

Methods described herein, including those with reference to one or more flowcharts, may be performed by one or more processing devices (e.g., smartphone, computer, playback device, etc.). The methods may include one or more operations, functions, or actions as illustrated in one or more blocks. Although the blocks are illustrated in sequential order, these blocks may also be performed in parallel, and/or in a different order than the order disclosed and described herein. Also, the various blocks may be combined into fewer blocks, divided into additional blocks, and/or removed based upon a desired implementation. Dashed lines may represent optional and/or alternative steps.

II. Example Multimodal Stimulation System

Neuromodulation via brain entrainment to a rhythmic stimulus may be more effective if several inputs to the brain are being utilized simultaneously. However, cross-sensory stimulus pairs may have different physical transmission and physiological transduction times, which may result in discrepancies in relative processing latencies in the order of tens of milliseconds. The brain may then perform “temporal recalibration” to make the perceptions coherent, but the neural bases of such operations are only recently being uncovered. Nonetheless, a phase/time difference between inputs may change the entrainment effect on the brain.

Therefore, the modulation parameters in the multiple inputs should be coordinated to produce maximum effect by their combination. For example, since light travels faster than sound, and since the optical pathway in the brain is more direct to the cortex than the auditory pathway in the brain, it is known that a flashing light should precede a modulated sound in phase, to have both signals coincide (be phase aligned) in the cortex.

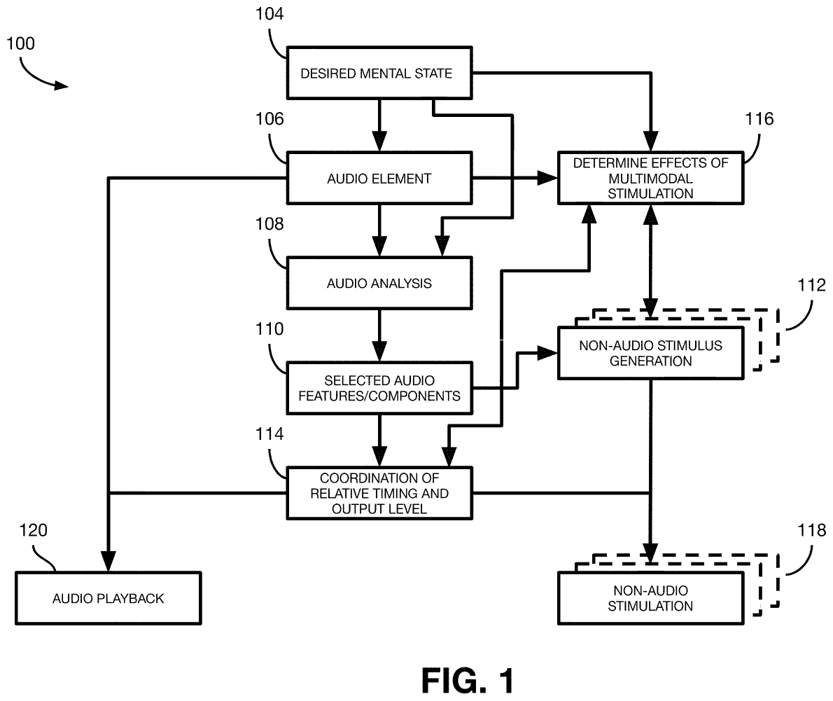

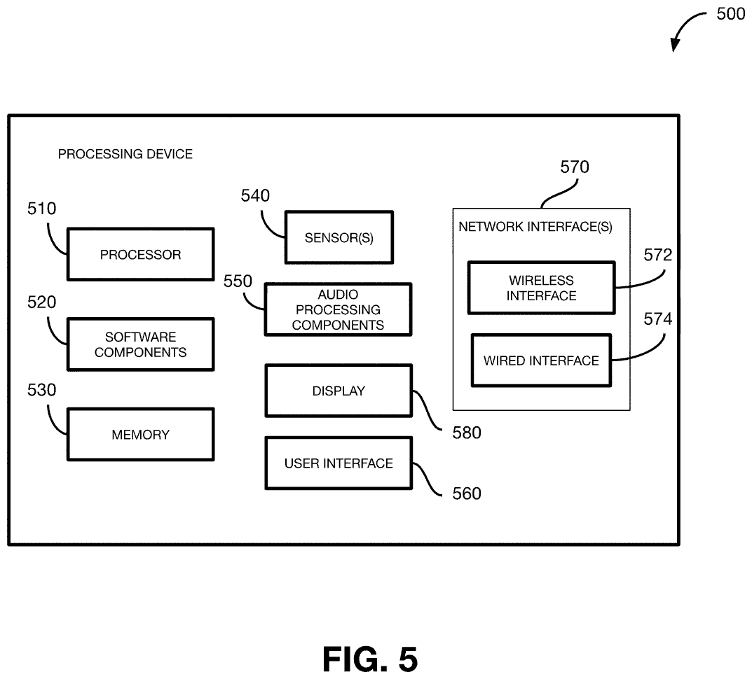

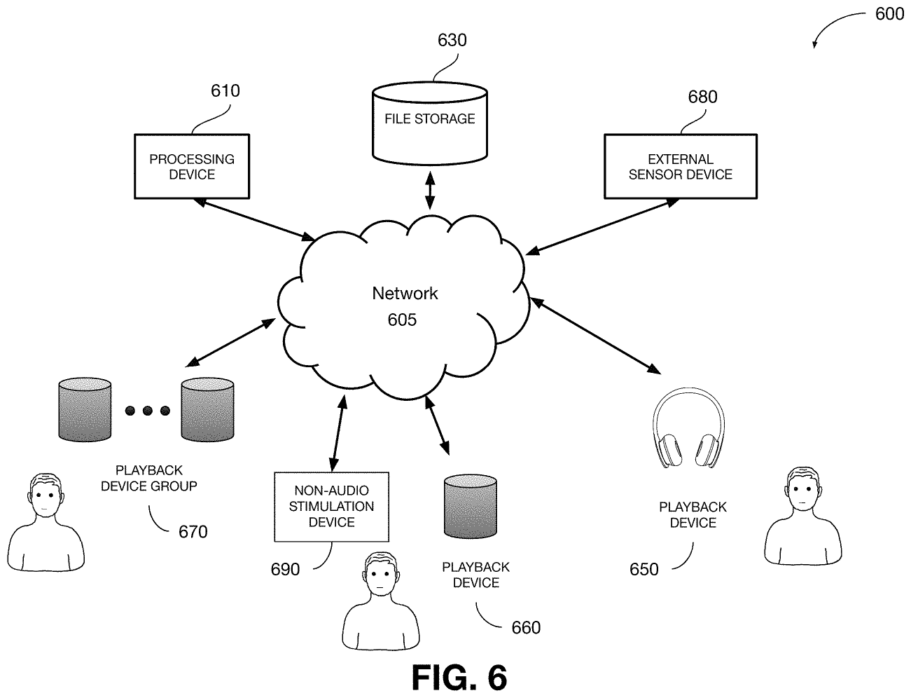

FIG. 1 depicts a flowchart illustrating a method 100 for coordinating modulation across multiple input modalities to the central nervous system to effectively induce and/or modify mental states by noninvasive brain stimulation. The method 100 may include one or more operations, functions, or actions as illustrated in one or more blocks 104-120. Although the blocks are illustrated in sequential order, these blocks may also be performed in parallel, and/or in a different order than the order disclosed and described herein. Also, the various blocks may be combined into fewer blocks, divided into additional blocks, and/or removed based upon a desired implementation. The method 100 may be implemented by one or more processing devices such as the processing device of FIG. 5 and/or the one or more processing devices shown in FIG. 6. The method 100 may increase the combined effect of the multiple input modalities on entrainment to produce a desired mental state for a user. In addition, combining audio and non-audio stimulation may be used to increase the neuromodulation effect beyond upper limits of what would be acceptable (e.g., aesthetically or physiologically) to a user for a single stimulation modality (e.g., audio). Once the input modalities (i.e., locations on the body and thus transmission latencies to the brain) are identified, predetermined and/or dynamic phase parameters may be used to coordinate the time of arrival of the signals to the brain.

The method 100 may be initiated on a processing device such as, for example, the processing device of FIG. 5, which may include one or more of a smartphone, laptop, computer, playback device, etc. In block 104, an indication of a desired mental state of a user is received at the processing device. The desired mental state may be selected explicitly (e.g. by the user) or may be selected automatically based on one or more parameters (e.g., an application that infers that a user wants to go to sleep due to the time of day, etc.). Non-limiting examples of a desired mental state may include focus, relax, sleep, and meditate. Each of these example desired mental states may be further distinguished by a target activity and duration. For example, focus may be distinguished by deep work, creative flow, study and read, and light work; relax may be distinguished by chill, recharge, destress, and unwind; sleep may be distinguished by deep sleep, guided sleep, sleep and wake, and wind down; and meditate may be distinguished by unguided and guided. The duration of the mental state may be specified, for example, by a time duration (e.g., minutes, hours, etc.), or a duration triggered by an event (e.g., waking, etc.). The indication may be received via a user interface on a processing device such as, for example, through an interface on the Brain.fm™ application executing on an iPhone™ or Android™ device. Alternatively and/or additionally, the indication may be received over a network from a different processing device.

In block 106, an audio element is received at the processing device from an audio source. The audio element may be selected by the user and/or the processing device. The desired mental state (e.g., received in block 104) may be used in the selection of the audio element. Additionally and/or alternatively, the audio element may be created with reference to the desired mental state and/or for other reasons (e.g., entertainment). The audio element may be, for example, a digital audio file retrieved by the processing device from local storage on the processing device or from remote storage on a connected device. In an example, the digital audio file is streamed to the processing device from a connected device such as a cloud server for an online music service (e.g., Spotify, Apple Music, etc.). In another example, the audio element may be received by the processing device from an audio input such as a microphone. The audio source can include, for example, an audio signal, digital music file, musical instrument, or environmental sounds. The audio element can be in the form of one or more audio elements read from a storage medium, such as, for example, an MP3 or WAV file, received as an analog signal, generated by a synthesizer or other signal generator, or recorded by one or more microphones or instrument transducers, etc. The audio elements may be embodied as a digital music file (.mp3, .wav, .flac, among others) representing sound pressure values, but could also be a data file read by other software which contains parameters or instructions for sound synthesis, rather than a representation of sound itself. The audio elements may be individual instruments in a musical composition, groups of instruments (bussed outputs), but could also be engineered objects such as frequency sub-bands (e.g., bass frequencies vs treble frequencies). The content of the audio elements may include music, but also non music such as environmental sounds (wind, water, cafe noise, and so on), or any sound signal such as a microphone input.

In an example embodiment, to achieve better brain stimulation, a wide variety of audio elements may be used, which may span different or complementary portions of the audio frequency spectrum, or cover a broad range of the spectrum. Accordingly, the audio elements may be selected such that they have a wide (i.e., broadband) spectral audio profile—in other words, the audio elements can be selected such that they include many frequency components. For example, the audio elements may be selected from music composed from many instruments with timbre that produces overtones across the entire range of human hearing (e.g., 20-20 kHz).

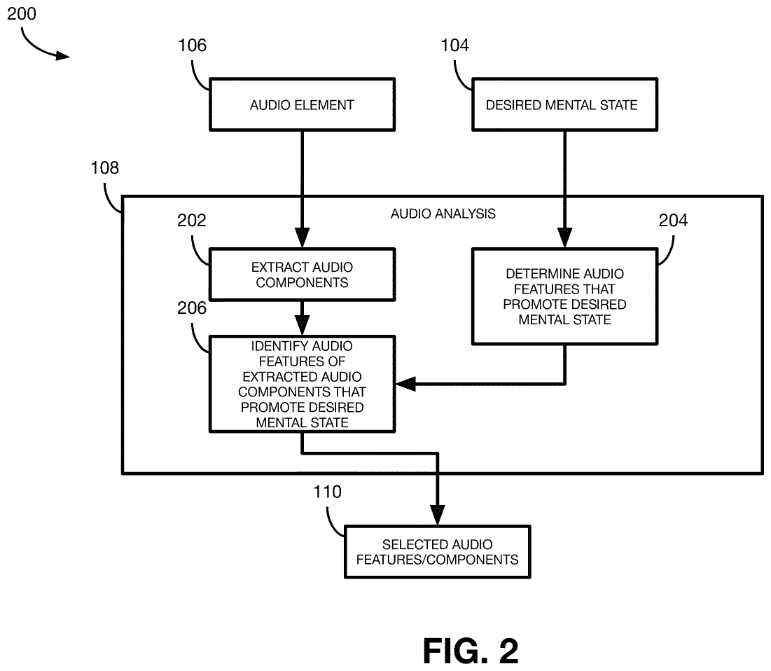

In block 108, the received audio may be analyzed to identify and/or determine one or more features/characteristics of the audio element. One or more aspects of block 108 are further discussed with respect to FIG. 2.

In block 110, features/components of the received audio that are identified and/or determined are extracted from the audio signal. The features/components may be simple (e.g., beat markers) or they may be more complex (e.g., extracted instruments, sub-band envelopes, modulation spectra, etc.).

In blocks 112, non-audio stimulus for use in one or more non-audio stimulation devices may be generated using the extracted audio features/components. This process may use information such as the type and/or location of each of the one or more non-audio stimulation devices and the desired mental state to generate the non-audio stimulus. This information may be either determined implicitly (e.g., from received audio features) or received explicitly (e.g., from the user or program). Information about the desired mental state may be used to guide non-audio stimulus generation. For example, if the desired mental state is sleep, the shape of a tactile waveform may be adjusted to be more soothing than a tactile stimulus for exercise. Many non-audio stimulus types may be created and used together with or without the original audio.

In block 114, relative timing (e.g., phase of modulation across modalities) and output level across the multiple stimuli may be coordinated. The relative timing may be based on, at least, location/position information of the one or more non-audio stimulation devices and/or the one or more audio playback devices. For example, a phase shift applied to a vibration device on a user's ankle may be greater than a phase shift applied to a similar device on the head based on how long the stimulus from the vibration device takes to reach the cortex. In addition, waveform shape and/or other signal parameters may be different from audio based on the non-audio stimulation device and sensory modality. For example, an envelope of an audio signal may be extracted and/or determined. The envelope may follow the music, or it may be shaped by one or more instruments' attack sustain decay release (ASDR) envelope. A waveform shape most effective on the non-audio modality may be different (e.g., triangle wave, sawtooth, etc.) than what is effective for an audio modality. In some examples, it may be beneficial to follow the timing of the audio modulation without exactly copying the shape of the envelope/waveform.

In block 116, a determination of effects of multimodal stimulation may be used to determine and/or adjust the relative timing of block 114. The determination may be based on, for example, one or more of a model/rules or sensor data. In an example, the model/rules of the effects of multimodal stimulation may be simple. For example, a model/rules may include ensuring rhythmic stimuli are synchronized by penalizing for more rather than less peaks (local envelope maxima). In another example, the model/rules may be complex. For example, the model/rules may be based on a research-based brain model of neural oscillations with effects of stimulus history or memory.

In another example, sensor data may be used in addition to or as a replacement of a model as long as the sensor data value is a truthful indicator (even indirectly) of the desired mental state (and thus can be used to guide the coordinating of the multiple stimulation signals). One difference from a model is that in the case of sensor data the parameter optimization process may need to prioritize smoothness and efficiency, so as not to have the stimulus jump around in parameter space. This might produce context effects in human listeners that are not desirable. The sensor data may be, for example, biosensor data (e.g., heart rate, blood pressure, breathing) or it may be transformed or combined sensor data estimating mental states (e.g., stress score, focus estimates).

Through analysis of sensor data at block 116, coordination between the different stimulation signals and the properties of the non-audio stimulation may be optimized. For example, brainwave states may be determined via one or more of electroencephalogram (EEG) and magnetoencephalogram (MEG) data and modulation characteristics of the non-audio stimulus may be adjusted, including phase shift relative to the audio, but also waveform shapes, amplitudes, etc. across different stimulating modalities to have a desired impact on the brain. Varying the modulation characteristics of non-audio stimulation according to sensor data in addition to or instead of audio may enable the dynamic variation of only the non-audio modality to avoid disrupting aesthetics of music. Carrier frequencies in the non-audio modality (tactile carrier frequencies, or colors of light) may also be varied.

The output of block 116 may be feedback (e.g., error/control signals) provided to one or more of blocks 112 and block 114 (e.g., from a single value of an estimated effect to simulated EEG data). The feedback error/control signals may be used to modify timing and/or non-audio stimulus parameters. Solving for the desired model output (based on desired mental state) may be done with one or more machine learning (ML) methods such as gradient descent.

In blocks 118, non-audio stimulus may be generated by the one or more non-audio stimulation devices and delivered to the user. The one or more non-audio stimulation devices may be any type of device that delivers non-audio stimulation to a user. For example, the one or more non-audio stimulation devices may include a wearable device that provides vibrotactile stimulation (e.g., on a wrist or ankle), a chair, bed, or other active furniture, brightness modulation of a screen, a transcranial electrical current stimulation device, and a one or more lights for photo-stimulation.

In block 120, audio stimulus may be generated by one or more audio devices and delivered to the user via an audio playback device. It should be noted that blocks 118 and 120 may be used concurrently (i.e., multimodal entrainment), block 118 may be used without block 120 (i.e., unimodal non-audio entrainment), and block 120 may be used without block 118 (i.e., unimodal audio entrainment). The flexibility of turning on and off either modality provides a number of benefits for users. For example, a user may wear a vibrating wristband synced to an audio output and may be able to mute the audio temporarily but still get an ongoing benefit of the tactile modulation.

FIG. 2 depicts an example flowchart 200 illustrating details of the audio analysis performed in block 108 and may include one or more additional steps. At block 202, one or more audio components are extracted from the received audio element 106. These audio components may include frequency sub-bands, instruments (e.g., extracted from a mix), or any other part which may be separated out from the audio, or feature extracted from the audio.

At block 204, one or more audio features that promote the desired mental state may be determined. These one or more audio features may be based on a user model that may prescribe regions in the modulation-characteristic space that are most effective for a desired mental state. The user model may define predicted efficacy of music as a function of dimensions such as modulation rate, modulation depth, audio brightness, audio complexity, or other audio features. The user model may be based on prior research that relates modulation characteristics to mental states. For example, if the user says they have ADHD and are of a particular age and gender, then the user model may incorporate this information to determine desired modulation characteristics for a particular target mental state of the user. The determination may, for example, be based on a stored table or function which is based on prior research about ADHD (e.g., users with ADHD require a relatively high modulation depth). Another non-limiting example for defining and/or modifying a user model may be based on reference tracks and ratings provided by a user. The reference tracks may be analyzed to determine their modulation characteristics. The determined modulation characteristics along with the ratings of those tracks may be used to define or modify the user model.

In an example, the user model may be updated over time to reflect learning about the user. The user model may also incorporate an analysis of various audio tracks that have been rated (e.g., {for effectiveness {focus, energy, persistence, accuracy}, or satisfaction}, positively or negatively). The inputs to generate a user model may include ratings (e.g., scalar (X stars), binary (thumbs up/down)), audio characteristics (e.g., modulation characteristics, brightness, etc.) For example, a user known to have ADHD may initially have a user model indicating that the target audio should have higher modulation depth than that of an average target track. If a user subsequently provides a reference track with a positive indication, and it is determined that the reference track has a low modulation depth (e.g., 0.2 out of 1), then the target modulation depth may be updated in the user model (e.g., to an estimate that a low depth is optimal). If the user subsequently provides three more reference tracks with positive indications, and it is determined that the tracks have modulation depths of 0.8, 0.7, and 0.9, then the target modulation depth may be further updated in the user model (e.g., reverting to an estimate that a high depth is optimal). In this example, the user model represents estimated effectiveness as a function of modulation depths from 0-1.

The user model may predict ratings over the modulation characteristic space. For example, if each input track is a point in high-dimensional space (e.g., feature values) each of which has been assigned a color from blue to red (e.g., corresponding to rating values); then the prediction of ratings may be determined by interpolating across known values (e.g., target input tracks) to estimate a heatmap representation of the entire space. In another example, regions of the space may be predicted to contain the highest rating values via linear regression (i.e., if the relationships are simple) or machine learning techniques (e.g., using classifiers, etc.).

The user model may be distinctive both in terms of the features used (e.g., modulation features relevant to effects on the brain and performance, rather than just musical features relevant to aesthetics) and in terms of the ratings, which may be based on effectiveness to achieve a desired mental state such as, for example, productivity, focus, relaxation, etc. rather than just enjoyment.

The user model may be treated like a single reference input track if the output to the comparison is a single point in the feature space (e.g., as a “target”) to summarize the user model. This may be done by predicting the point in the feature space that should give the highest ratings and ignoring the rest of the feature space. In this case the process surrounding the user model may not change.

In some examples, a user model may not be required. For example, if multiple reference tracks and ratings are provided as input, the processing device may forgo summarizing them as a model and instead work directly off this provided data. For example, each library track may be scored (e.g., predicted rating) based on its distance from the rated tracks (e.g., weighted by rating; being close to a poorly rated track is bad, etc.). This may have a similar outcome as building a user model but does not explicitly require a user model.

In an example where only one reference track is used as input, it may be desirable to forgo a user model altogether, and directly compare the reference track to one or more target tracks. This is similar to a user model based only on the one reference track. If the reference track and the one or more target tracks are compared directly, they may be represented in the same dimensional space. Thus, the audio analysis applied to the reference track should result in an output representation that has the same dimensions as the audio analysis that is applied to the one or more target tracks.

In block 206, the one or more audio features may be identified from the extracted audio components. For example, it might be known (via user model or not) that modulations in the range <=1 Hz, with a particular waveform and depth, are most effective for inducing sleep. Given a user's goal of wanting to sleep (block 104), the determination is made in block 204 to use modulation rates of <=1 Hz of a particular waveform and depth. In block 206 the system searches for which audio components extracted from the audio element (block 106) best match the modulation properties targeted by block 204. The audio features that contain modulation may include the envelope of the audio waveform of the broadband or sub-band signals or other audio parameters. For example, modulation may be calculated in RMS (root mean square energy in signal); loudness (based on perceptual transform); event density (complexity/business); spectrum/spectral envelope/brightness; temporal envelope (‘out-line’ of signal); cepstrum (spectrum of spectrum); chromagram (what pitches dominate); flux (change over time); autocorrelation (self-similarity as a function of lag); amplitude modulation spectrum (how is energy distributed over temporal modulation rates); spectral modulation spectrum (how is energy distributed over spectral modulation rates); attack and decay (rise/fall time of audio events); roughness (more spectral peaks close together is rougher; beating in the ear); harmonicity/inharmonicity (related to roughness but calculated differently); and/or zero crossings (sparseness). Extraction of these features may be performed, for example, as multi-timescale analysis (different window lengths); analysis of features over time (segment-by-segment); broadband or within frequency sub-bands (i.e., after filtering); and/or second order relationships (e.g., flux of cepstrum, autocorrelation of flux).

In an example case, the desired mental state (block 104) might be Focus, and this might be determined (block 204) to require modulation rates of 12-20 Hz with a peaked waveform shape. The input audio element (block 106) is decomposed into audio components (block 202) including sub-band envelopes, cepstra, and other features; in this example case, among these components there is a particular high-frequency sub-band's envelope which contains modulation energy with a strong component at 14 Hz. This audio component is identified in block 206 and is then used to create the non-audio stimulation. The output of block 206 may be the selected audio features/components of block 110.

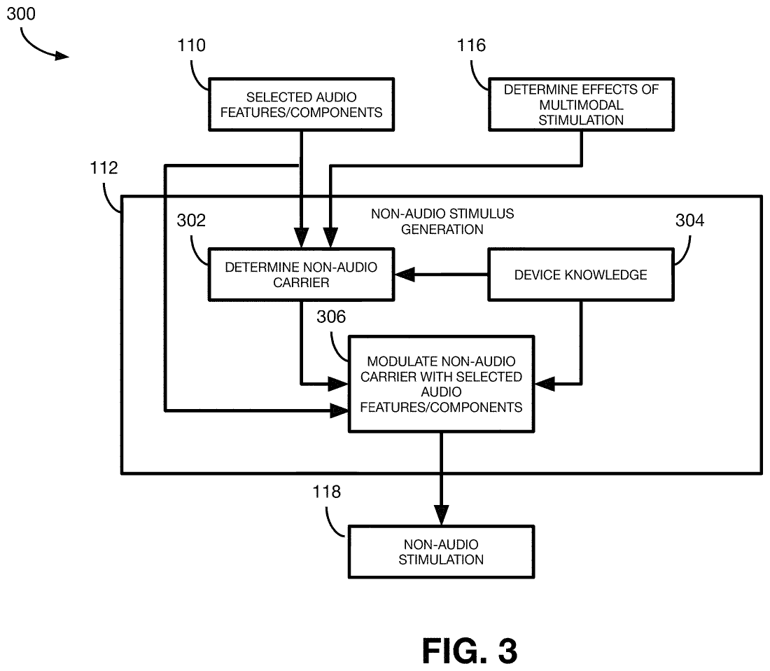

FIG. 3 depicts an example flowchart 300 illustrating details of the generation of non-audio stimuli performed in block 112. The selected audio features/components of block 110 can be one input to block 112. Another input to block 112 can be feedback (e.g., error/control signals) provided to block 112 and may be simple or complex (e.g., from a single value of an estimated effect to simulated EEG data). The feedback error/control signals may be used to modify timing and or non-audio stimulus parameters.

In block 302, a non-audio carrier may be determined based on one or more of device information from block 304, the selected audio features/components of block 110, and feedback from block 116. For example, if the non-audio stimulation device is a haptic wristband and the extracted audio features are rapid modulations, when determining the non-audio carrier (block 302), there may be constraints on the range of vibratory frequencies which should be used by the wristband to carry the modulations extracted from the audio (e.g., based on the rate of modulation, waveshape, and/or other factors). Further, the range of modulated frequencies may be modified based on a determination of the effects of the multimodal stimulation (block 116). In block 306, the non-audio carrier may be modulated with the selected audio features/components (block 110) to produce a signal that may be transmitted to a non-audio stimulation device which generates non-audio stimulation from the signal (block 118).

In an example, the audio analysis performed in block 108 of the audio element received in block 106 may identify characteristics that promote a desired mental state in block 104 (e.g., focus) in a high-frequency sub-band envelope as shown in FIG. 2. For example, very regular and pronounced 16 Hz envelope modulations (desirable for a focused mental state) may have been found in a particular high-frequency sub-band due to a fast bright instrument (e.g., hi-hat). These 16 Hz envelope modulations may comprise the selected audio features/components 110.

In an example, a low-frequency (e.g., 30-300 Hz) sub-band of the same audio element may be determined to be the non-audio carrier determined in block 302. In another example, block 302 may include the generation of a non-audio carrier. For example, the non-audio carrier may be one or more stable vibration rates tuned to a sensitivity of the relevant region of the body, or may be a shifting vibrational rate that follows the dominant pitch in the music. Information about the one or more non-audio devices in block 304 may be used to generate an effective output signal. In an example, a tactile device (e.g., vibrating wristband) may be known to work well between 30 Hz and 300 Hz, so the non-audio stimulus may be created within this passband.

In an example, different portions of audio frequency may be mapped to different outputs in the non-audio sensory modality. For example, modulation in high frequencies versus low frequencies may be mapped to different parts of the visual field (which would stimulate left vs right hemispheres selectively), or wrist vs ankle stimulation. There often many modulation rates in a piece of audio. In music used primarily for entrainment this may be deliberate (e.g., to target relax and sleep rates simultaneously). This characteristic may be transferred to the non-audio modality either by combining the rates into a complex waveform or by delivering the different rates to different sub-regions of the non-audio modality (e.g., visual field areas, wrist vs ankle, etc.).

Instead of the non-audio signal simply following the audio envelope, desired modulation rates may be extracted and/or determined from the audio envelope and used to generate the non-audio stimulus. For example, a piece of audio may be complex music with added modulation at 16 Hz for focus. The audio envelope from the selected audio features/components of block 110 may have a strong 16 Hz component but will also contain other aspects of the audio. The system may determine that 16 Hz is the dominant modulation rate and drive non-audio stimulation with a 16 Hz simple wave (e.g., sine, square, etc.). Multiple modulation rates may be extracted and/or determined from the audio, for example, in separate frequency bands or the same frequency band (i.e., by decomposition of cochlear envelopes).

In contrast to existing systems that analyze audio and produce non-audio stimulation (e.g., music visualizers), the system does not aim to match the music in every aspect. Instead, regular rhythmic stimulus may be generated to drive entrainment at a particular modulation rate. While the phase of the modulation must be tightly controlled across the two sensory modalities, the signals themselves may be quite different. For example, tactile stimulation may be generated by modulating a carrier such as a low frequency suitable for tactile stimulation (e.g., 70 Hz) by the entraining waveform (e.g., a 12 Hz triangle wave phase-locked to the 12 Hz component in the audio envelope). In another example, the non-audio modality may not be directly driven by the cycle-by-cycle amplitude of the audio, but instead the system may find the desired rate and phase of modulation in the audio, align the non-audio signal to it, and drive the brain strongly at that rate regardless of the audio. For example, “weak” beats in audio may be ignored in favor of having the non-audio signal stimulate to a regular amplitude on each cycle.

Perceptual coherence (i.e., information from the different senses represents the same event in the world) may be improved by using low frequencies in the music, or subharmonics of the dominant fundamental frequencies. Perceptual coherence is desirable not only for aesthetic reasons, but also functional reasons (i.e., less distracting to have one thing versus two things going on) and neural reasons (i.e., representation in the brain coincides; likely to enhance entrainment).

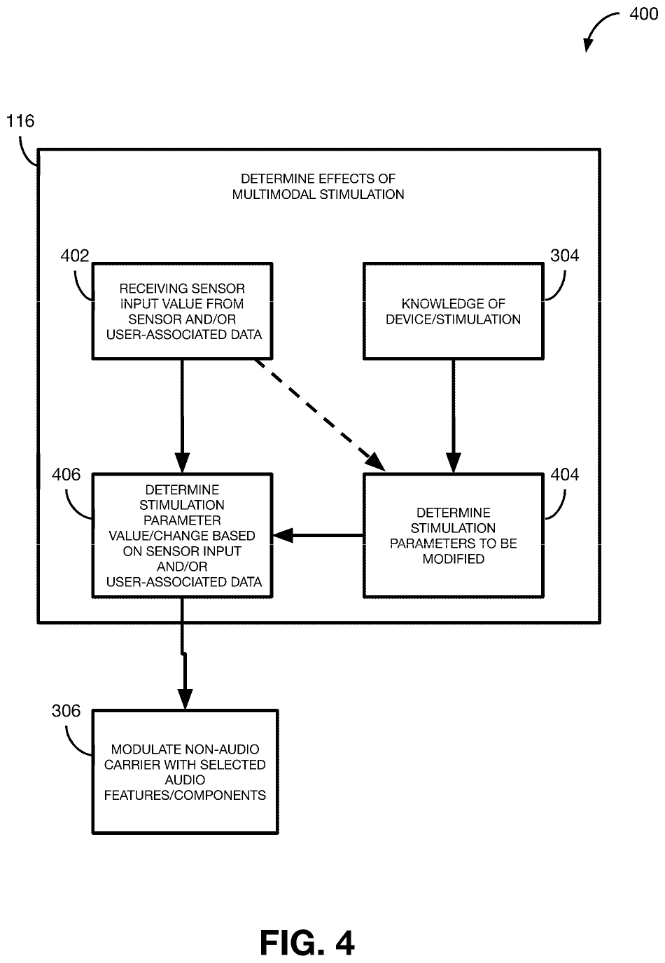

FIG. 4 depicts a flowchart 400 illustrating details of an example using sensor data to determine effects of multimodal stimulation as performed in block 116. In some examples, sensors may inform the system about the user's mental state, brain activity, user behavior, or the like. The sensor data should be responsive to, directly or indirectly, changes in the multimodal stimulation. At block 402, a sensor-input value may be received from a sensor. The sensor may be on the processing device or it may be on an external device and data from the sensor may be transferred to the processing device. In one example, the sensor on a processing device, such as an accelerometer on a mobile phone, may be used to determine how often the phone is moved and may be a proxy for productivity. In another example, the sensor on an activity tracker (external device), for example an Oura ring or Apple watch, may be used to detect if the user is awake or not, how much they are moving, etc.

In some embodiments, the sensors may be occasional-use sensors responsive to a user associated with the sensor. For example, a user's brain response to the relative timing between light and sound modulation may be measured via one or more of EEG and MEG during an onboarding procedure which may be done per use or at intervals such as once per week or month.

In some embodiments, behavioral/performance testing may be used to calibrate the sensors and/or to compute sensor-input values. For example, a short experiment for each individual to determine which timing across stimulation modalities is best for the user by measuring performance on a task. Similarly, external information may be used to calibrate the sensors and/or to compute sensor-input values. For example, weather, time of day, elevation of the sun at user location, the user's daily cycle/circadian rhythm, and/or location. In an example case, for a user trying to relax, a sensor might read a user's heart rate variability (HRV) as an indicator of arousal/relaxation, and this feedback signal may be used to optimize the parameters of the non-audio stimulus and the coordination of the two stimulation modalities. The external information of the time of day may be taken into account by the algorithm predicting arousal from HRV, in that the relationship between them varies based on time of day. Of course, each of these techniques may be used in combination or separately. A person of ordinary skill in the art would appreciate that these techniques are merely non-limiting examples, and other similar techniques may also be used for calibration of the sensors.

In example embodiments, the sensor-input value may be obtained from one or more sensors such as, for example, an accelerometer (e.g., phone on table registers typing, proxy for productivity); a galvanic skin response (e.g. skin conductance); video (user-facing: eye tracking, state sensing; outward-facing: environment identification, movement tracking); microphone (user-sensing: track typing as proxy for productivity, other self-produced movement; outward-sensing: environmental noise, masking); heart rate monitor (and heart rate variability); blood pressure monitor; body temperature monitor; EEG; MEG (or alternative magnetic-field-based sensing); near infrared (fnirs); or bodily fluid monitors (e.g., blood or saliva for glucose, cortisol, etc.). The one or more sensors may include real-time computation. Non-limiting examples of a real-time sensor computation include: the accelerometer in a phone placed near a keyboard on table registering typing movements as a proxy for productivity; an accelerometer detects movements and reports user started a run (e.g. by using the CMMotionActivity object of Apple's iOS Core ML framework), and microphone detects background noise in a particular frequency band (e.g., HVAC noise concentrated in bass frequencies) and reports higher levels of distracting background noise.

The received sensor-input value may be sampled at pre-defined time intervals, or upon events, such as the beginning of each track or the beginning of a user session or dynamically on short timescales/real-time: (e.g., monitoring physical activity, interaction with phone/computer, interaction with app, etc.).

In an example embodiment, block 402 may include receiving user-associated data in addition and/or alternatively to the previously described sensor-input value from the sensor (not shown). Alternatively, the block 402 may include receiving only the sensor-input value or user-associated data.

In example embodiments, user-associated data may include self-report data such as a direct report or a survey, e.g., ADHD self-report (ASRS survey or similar), autism self-report (AQ or ASSQ surveys or similar), sensitivity to sound (direct questions), genre preference (proxy for sensitivity tolerance), work habits re. music/noise (proxy for sensitivity tolerance), and/or history with a neuromodulation. Self-report data may include time-varying reports such as selecting one's level of relaxation once per minute, leading to dynamic modulation characteristics over time in response. User-associated data may include behavioral data/attributes such as user interests, a user's mental state, emotional state, etc. Such information may be obtained from various sources such as the user's social media profile. User-associated data may include factors external to but related to the user such as the weather at the user's location; the time after sunrise or before sunset at the user's location; the user's location; or whether the user is in a building, outdoors, or a stadium.

At block 404, one or more parameters of coordination between the multiple stimulation modalities (relative timing/phase, relative power/depth, etc.) and/or parameters of the non-audio stimulation (i.e., modulation-characteristic values such as rate, waveform shape, etc.) may be determined. This determining may be based on the stimulation being provided (audio and/or non-audio) or predetermined based on knowledge of the device and/or stimulation (e.g., from block 304). For example, in a case where light and sound are being delivered to the user, two determined stimulation parameters in block 404 might be the relative phase between light and sound modulation, and the depth of light modulation; but in a case where only light is being delivered (uni-modal stimulation), the determined stimulation parameters in block 404 might instead be the depth of light modulation alone. The sensor input used for feedback in block 402 may also contribute to determining which stimulation parameters should be selected for adjustment by the system. For example, noisy data from a sensor might invalidate the device knowledge from block 304_as to which stimulation parameters the system expected to use; after receiving real data from the sensor from block 402, the system may override the determination it would otherwise have made in block 404. In block 406, the determined stimulation parameters may be adjusted by the system via a feedback signal. The modified stimulation is delivered to the user which may result in a modified user state and sensor data, and thereby closing a feedback loop.

The mapping of sensor-input values and stimulation parameters may correlate each sensor-input value to a respective stimulation parameter value. For example, in a case where the sensor is an EEG headset measuring neural phase-locking (synchrony, entrainment), and a determined stimulation parameter is phase across light and sound modulation, a mapping may exist which enforces that, if neural entrainment is low, the phase difference between light and sound is shifted (i.e., “increased,” but phase is circular so an increase becomes a decrease after 180 degrees). If neural entrainment is high, the phase difference may not be changed as much or at all. Such a mapping may be based on absolute sensor values, on values relative to the user or other users (e.g., zero-mean data, % of max), and/or on changes in values (e.g., time-derivative of sensor data). The mapping may be based on a predetermined or real-time computed map. Non-limiting examples of mappings include: a phone with an accelerometer that detects movement and reports an estimate of user productivity and mapping this productivity estimate to light modulation depth such that the level of non-audio modulation increases if estimated productivity slows down. Other examples exist. The mapping may be stored in a data table as shown in the example below in table 1 or stored as a function, such as, for example, f(x)=x² where x is the sensor-input value and f(x) is the modulation characteristic value.

TABLE 1

| Sensor input values | stimulation parameters |
|---|---|
| (Neural Phase- | (shift in phase difference |
| Locking value, | between light and |
| power) | sound modulation, deg/min) |
| 20 | 90 |
| 30 | 80 |
| 40 | 70 |
| 50 | 60 |
| 60 | 50 |
| 70 | 40 |
| 80 | 20 |
| 90 | 10 |
| 100 | 0 |
| 110 | 0 |
| 120 | 0 |
| 130 | 0 |
| 140 | 0 |
| 150 | 0 |
| 160 | 0 |
| 170 | 0 |
| 180 | 0 |
| 190 | 0 |
| 200 | 0 |

In an example, modulation rate (e.g., of all stimulation modalities), phase (i.e., difference between stimulation modalities), depth (i.e., of one or more stimulation modalities, or the relative levels between them), and waveform shape (i.e., of the non-audio stimulation modality) may be four non-exclusive modulation characteristics (i.e., stimulation parameters). Modulation rate may be the speed of the cyclic change in energy, and may be defined, for example, in hertz. Phase is the particular point in the full cycle of modulation, and may be measured, for example, as an angle in degrees or radians. Depth may indicate the degree of amplitude fluctuation in the audio signal. In amplitude modulation, depth may be expressed as a linear percent reduction in signal power or waveform envelope from peak-to-trough, or as the amount of energy at a given modulation rate. Waveform may express the shape of the modulation cycle, such as a sine wave, a triangle wave or some other custom wave. These modulation characteristics may be extracted and/or determined from the broadband signal or from sub-bands after filtering in the audio-frequency domain (e.g., bass vs. treble), by taking measures of the signal power over time or by calculating a waveform envelope (e.g., the Hilbert envelope).

A stimulation protocol may provide one or more of a modulation rate, phase, depth and/or waveform for the modulation to be applied to audio data that may be used to induce neural stimulation or entrainment. Neural stimulation via such a stimulation protocol may be used in conjunction with a cochlear profile to induce different modes of stimulation in a user's brain. A stimulation protocol can be applied to audio and/or non-audio stimulation. For example, a stimulation protocol for modulated light would have the same description as that for audio, describing modulation rate, phase, and depth, over time (only, of illumination/brightness rather than sound energy).

At block 306, one or more of the relative timing and characteristics of non-audio output may be adjusted based on the one or more stimulation parameter values determined in 406. The one or more of the relative timing and characteristics of non-audio output may be adjusted by varying one or more of a modulation rate, phase, depth and/or waveform in real-time, at intervals, or upon events, such as the beginning of each track or the beginning of a user session. As described above, the adjustment may be in the form of feedback (e.g., error/control signals) to one or more of block 112 and block 114. If some or all of these parameters are described as a stimulation protocol, these adjustments could take the form of modifying the stimulation protocol.

FIG. 5 shows a functional block diagram of an example processing device 500 that may implement the methods previously described with reference to FIGS. 1-4. The processing device 500 includes one or more processors 510, software components 520, memory 530, one or more sensor inputs 540, audio processing components (e.g. audio input) 550, a user interface 560, a network interface 570 including wireless interface(s) 572 and/or wired interface(s) 574, and a display 580. The processing device may further optionally include audio amplifier(s) and speaker(s) for audio playback. In one case, the processing device 500 may not include the speaker(s), but rather a speaker interface for connecting the processing device to external speakers. In another case, the processing device 500 may include neither the speaker(s) nor the audio amplifier(s), but rather an audio interface for connecting the processing device 500 to an external audio amplifier or audio-visual playback device. The processing device may further optionally include non-audio stimulation elements such as, for example, vibration bed, an electrical brain-stimulation element, one or more lights, etc. In another case, the processing device 500 may not include non-audio stimulation elements, but rather an interface for connecting the processing device 500 to an external stimulation device.

In some examples, the one or more processors 510 include one or more clock-driven computing components configured to process input data according to instructions stored in the memory 530. The memory 530 may be a tangible, non-transitory computer-readable medium configured to store instructions executable by the one or more processors 510. For instance, the memory 530 may be data storage that may be loaded with one or more of the software components 520 executable by the one or more processors 510 to achieve certain functions. In one example, the functions may involve the processing device 500 retrieving audio data from an audio source or another processing device. In another example, the functions may involve the processing device 500 sending audio and/or stimulation data to another device (e.g., playback device, stimulation device, etc.) on a network.

The audio processing components 550 may include one or more digital-to-analog converters (DAC), an audio preprocessing component, an audio enhancement component or a digital signal processor (DSP), and so on. In one embodiment, one or more of the audio processing components 550 may be a subcomponent of the one or more processors 510. In one example, audio content may be processed and/or intentionally altered by the audio processing components 550 to produce audio signals. The produced audio signals may be further processed and/or provided to an amplifier for playback.

The network interface 570 may be configured to facilitate a data flow between the processing device 500 and one or more other devices on a data network, including but not limited to data to/from other processing devices, playback devices, stimulation devices, storage devices, and the like. As such, the processing device 500 may be configured to transmit and receive audio content over the data network from one or more other devices in communication with the processing device 500, network devices within a local area network (LAN), or audio content sources over a wide area network (WAN) such as the Internet. The processing device 500 may also be configured to transmit and receive sensor input over the data network from one or more other devices in communication with the processing device 500, network devices within a LAN or over a WAN such as the Internet. The processing device 500 may also be configured to transmit and receive audio processing information such as, for example, a sensor-modulation-characteristic table over the data network from one or more other devices in communication with the processing device 500, network devices within a LAN or over a WAN such as the Internet.

As shown in FIG. 5, the network interface 570 may include wireless interface(s) 572 and wired interface(s) 574. The wireless interface(s) 572 may provide network interface functions for the processing device 500 to wirelessly communicate with other devices in accordance with a communication protocol (e.g., any wireless standard including IEEE 802.11a/b/g/n/ac, 802.15, 4G and 5G mobile communication standard, and so on). The wired interface(s) 574 may provide network interface functions for the processing device 500 to communicate over a wired connection with other devices in accordance with a communication protocol (e.g., IEEE802.3). While the network interface 570 shown in FIG. 5 includes both wireless interface(s) 572 and wired interface(s) 574, the network interface 570 may in some embodiments include only wireless interface(s) or only wired interface(s).

The processing device may include one or more sensor(s) 540. The sensors 540 may include, for example, inertial sensors (e.g., accelerometer, gyrometer, and magnetometer), a microphone, a camera, or a physiological sensor such as, for example, a sensor that measures heart rate, blood pressure, body temperature, EEG, MEG, Near infrared (fNIRS), or bodily fluid. In some example embodiments, the sensor may correspond to a measure of user activity on a device such as, for example, a smart phone, computer, tablet, or the like.

The user interface 560 and display 580 may be configured to facilitate user access and control of the processing device. Example user interface 560 include a keyboard, touchscreen on a display, navigation device (e.g., mouse), etc.

The processor 510 may be configured to receive a mapping of sensor-input values and stimulation parameters, wherein each sensor-input value corresponds to a respective modulation-characteristic value. The processor 510 may be configured to receive an audio input from an audio source (not shown), wherein the audio input comprises at least one audio element, each comprising at least one audio parameter.

The processor 510 may be configured to identify an audio-parameter value of the audio parameter. The processor 510 may be configured to receive a sensor input 540 from a sensor (not shown). The processor 510 may be configured to select from the mapping of sensor-input values and stimulation parameters, a modulation-characteristic value that corresponds to the sensor-input value. The processor 510 may be configured to generate an audio output (or other stimulus output) based on the audio-parameter value and the modulation-characteristic value. The processor 510 may be configured to play the audio output and/or non-audio stimulus output.

Aspects of the present disclosure may exist in part or wholly in, distributed across, or duplicated across one or more physical devices. FIG. 6 is a functional block diagram that illustrates one such example system 600 in which the present invention may be practiced. The system 600 illustrates several devices (e.g., computing device 610, audio processing device 620, file storage 630, playback device 650, 660, and playback device group 670) interconnected via a data network 605. The playback device 650, 660, and playback device group 670 may be the one or more non-audio stimulation devices and the one or more audio playback devices. Although the devices are shown individually, the devices may be combined into fewer devices, separated into additional devices, and/or removed based upon an implementation. The data network 605 may be a wired network, a wireless network, or a combination of both.

In some example embodiments, the system 600 may include an audio processing device that may perform various functions, including but not limited to audio processing. In an example embodiment, the system 600 may include a computing device 610 that may perform various functions, including but not limited to, aiding the processing by the audio processing device 620. In an example embodiment, the computing devices 610 may be implemented on a machine such as the previously described system 600.

In an example embodiment, the system 600 may include a storage 630 that is connected to various components of the system 600 via a network 605. The connection may also be wired (not shown). The storage 630 may be configured to store data/information generated or utilized by the presently described techniques. For example, the storage 630 may store the mapping of sensor-input values and stimulation parameters. The storage 630 may also store the audio output generated.

In an example embodiment, the system 600 may include one or more playback devices 650, 660 or a group of playback devices 670 (e.g. playback devices, speakers, mobile devices, etc.), and one or more non-audio stimulation devices 690. These devices may be used to playback the audio output and/or non-audio stimulus. In some example embodiments, a playback device may include some or all of the functionality of the computing device 610, the audio processing device 620, and/or the file storage 630. As described previously, a sensor may be based on the audio processing device 620 or it may be an external sensor device 680 and data from the sensor may be transferred to the audio processing device 620.

The neuromodulation via brain entrainment to a rhythmic sensory stimulus described above, whether unimodal or multimodal, may be used to assist in sleep, to aid in athletic performance, and in medical environments to assist patients undergoing procedures (e.g., anesthesia, giving birth, etc.).

III. Example Method of Use of Sensory Neuromodulation for Recovery from Anesthesia

Induction and emergence from anesthesia may be a difficult process for patients and healthcare workers, and long recovery times may limit the rate of care that may be provided. Difficulties around induction and emergence from general anesthesia are a burden on healthcare workers, and central to a patient's experience. Anxiety prior to a procedure, and confusion upon regaining consciousness, are common experiences that negatively affect both patients and staff. Presurgical anxiety may result in difficulty with intubation and longer presurgical delay periods, burdening nurses and slowing the pace of care. Post surgically, the duration and quality of a patient's recovery from anesthesia affects healthcare providers and patients, both of whom desire to minimize the time spent in the recovery room. Lengthy recovery periods may involve amnesic episodes, delirium, agitation, cognitive dysfunction or other emergence phenomenon, which place strain on patients and staff. Longer recoveries also place strain on the patient's caretaker (e.g., relatives waiting to take them home) and burden the healthcare facility, which may be limited in how quickly procedures may occur based on space available in the recovery area.

Perioperative music has been used effectively to control anxiety and pain associated with surgeries; however, the music is typically selected to be either relaxing or familiar, with no regard for how it drives brain activity. Conventional work has focused on the preoperative period and has not considered how stimulative music might be used to kickstart cognition postoperatively following emergence from anesthesia. As an example, stimulative music may be characterized as audio with a peak (e.g., or local maximum) in modulation energy (e.g., as measured by a modulation spectrum or similar representation) in the range of 12-40 Hz. Typical music does not contain distinct rhythmic events at rates above 12 Hz, and thus does not contain peaks at these higher rates. Examples of stimulative music include music made purposely to drive rhythmic neural activity (e.g., brain entrainment) at these high rates, such as, for example, the tracks Rainbow Nebula and Tropical Rush developed by Brain.fm. Binaural beats (a type of sound therapy that drives neural entrainment but does not contain such modulation in the signal itself) has been proposed for perioperative use, but for relaxation only rather than stimulation. Accordingly, it may be desirable to use the rhythmic stimulation described above for induction and emergence, and/or to provide stimulative music to aid recovery from the unconscious state.

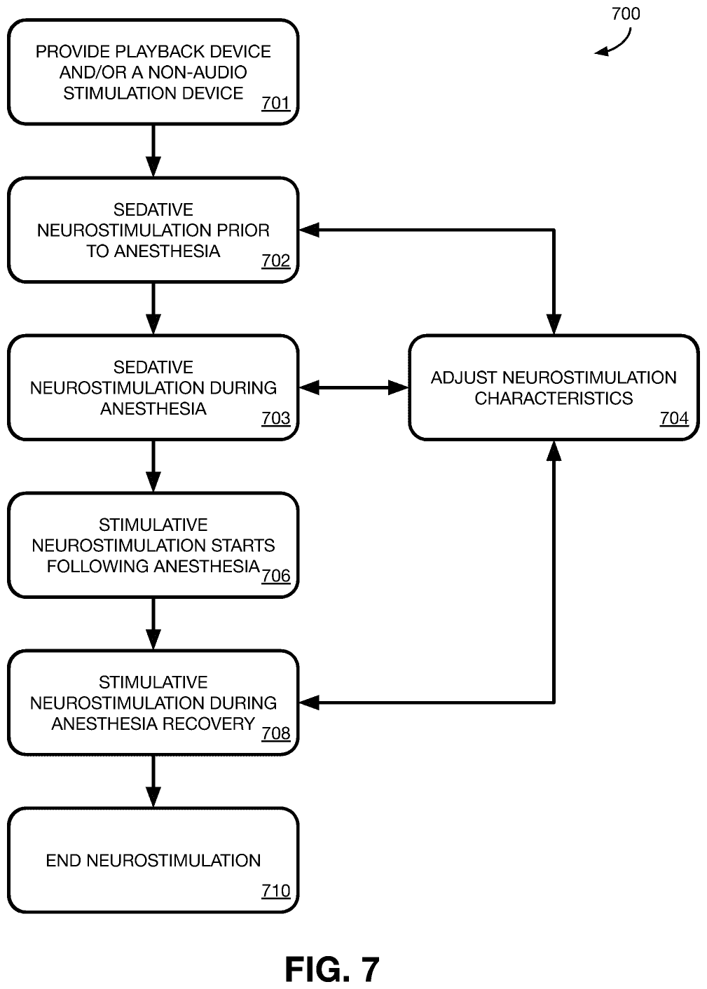

Referring now to FIG. 7, a flowchart illustrating a method 700 for using rhythmic stimulation to improve patient satisfaction and performance before, during, and after anesthesia is shown. Neuromodulation using rhythmic stimulation may reduce anxiety and improve relaxation during periods of induction and unconsciousness and may speed up emergence and recovery postoperatively.

In an example, one or more pieces of audio may be selected for playback at different points in the anesthesia process for sedative and/or stimulative properties. The audio may be delivered via one or more audio playback devices. In some examples, playback devices that permit a patient to maintain situational awareness while minimizing disturbances for caregivers and fellow patients is desired (e.g., bone-conduction headphones, pass-through headphones, nearfield speakers, etc.). As described above, accompanying non-audio stimulation may be delivered by one or more non-audio output devices (e.g., wearables, connected vibrating bed, lights, etc.) to further benefit the user. Audio and non-audio delivery may be accomplished via the same device, or different devices. Audio and non-audio stimulation files, instructions, programs, or other information needed to generate the stimulation (e.g., .mp3 file) may be stored on the stimulating device, or may be stored on a separate device and transmitted to the stimulation device. In an example, a pair of bone-conduction headphones may be connected and/or contain a memory card with a stimulating music track and a sedative music track. A button on the headphones may switch between the two tracks. Hospital staff may be instructed to press the button once when anesthesia is ceased following surgery and once again after the patient is discharged and returns their headphones. A similar example may use a vibrating wristband instead of headphones.

The audio and/or non-audio stimulation may be performed in sequence with the medical procedure and may be modulated in a desired way. In block 701, a patient may be provided a personal playback device (e.g., headphones) and/or a non-audio stimulation device (e.g., vibrating wrist band). In block 702, the patient may be given sedative audio stimulation (and/or non-audio stimulation) prior to administration of anesthesia. In an example, the audio and/or non-audio stimulation may be started just prior (e.g., less than 2 minutes) to administration of intravenous (IV) anesthesia to ensure that the audio and/or non-audio stimulation will be maximally novel and effective while the administration of anesthesia is being started (a highly anxiety-inducing event for many patients). The audio stimulation and/or non-audio stimulation may be modulated as desired. For example, some oscillations may be enforced while others may be dampened using uneven time signatures in music (e.g., 5/4 subdivided as 2-3-2-3). Additionally and/or alternatively, sedative audio and/or non-audio stimulation may also be administered during the procedure (i.e. while anesthesia is being administered) as indicated in block 703.

In block 704, one or more characteristics of the audio stimulation and/or non-audio stimulation may be adjusted prior, during, or after the procedure. For example, based on information obtained from one or more sensors, the characteristics of the audio and/or non-audio stimulation may be adjusted.

In block 706, once the procedure is finished and the administration of the anesthesia (e.g., through an IV) has stopped, the audio stimulation (and/or non-audio stimulation) may be switched to have a stimulative effect to assist in emergence and recovery from the anesthesia.

In block 708, as the patient recovers from anesthesia, audio and/or non-audio stimulation may continue, which may be responsive to the user's state via sensors (as in the previous stages before and during their procedure as indicated in block 704). For example, as the user's level of arousal increases, a patient may move more, which may be detected by accelerometers in their headphones; the detection of arousal (e.g., movement) may be a trigger to the stimulation protocol to modify the output (e.g., to decrease the volume level in the headphones so that volume is loudest when the patient is unconscious and less overbearing as the patient becomes aroused.

In block 710, the audio playback and/or non-audio stimulation may be ended, or the playback device (e.g., headphones) and/or stimulation device may be removed or disabled, when it is determined that the user is conscious and sufficiently recovered from anesthesia. This may be done manually by an operator (e.g., post-anesthesia care nurse, or the patient themselves) or automatically using input data from sensors to detect the patient's state and the playback device and/or non-audio stimulation device.

The one or more characteristics of the audio and/or non-audio stimulation (e.g., gain/depth, modulation, tempo, type of audio) may be modified manually by the patient and/or caregivers (e.g., when a patient is asleep) via, for example, a button and/or device such as a tablet. For example, a caregiver may manually switch the type of the audio and/or non-audio stimulation to stimulative once a procedure is finished.

Additionally or alternatively, the one or more characteristics of the audio and/or non-audio stimulation may be controlled automatically so that it is hands-free for the patient and/or caregiver. The automation may be accomplished using one or more methods, such as geolocation of a patient/device, WiFi, a physical sensor (e.g., in a bed), and an infrared (IR) sensor. These may be housed in the audio and/or non-audio stimulation device, or in separate devices. For example, the audio and/or non-audio stimulation may automatically switch to have a stimulative effect when the patient is unconscious and wake-up is desired (e.g., following cessation of anesthesia). Gain/depth of the audio stimulation may be controlled automatically (e.g., audio may be at its highest volume when a patient is most unconscious and ramps down over time). This may increase the effectiveness of the audio stimulation while a patient is under anesthesia as the brain may have a reduced firing rate and response to auditory stimuli is much weaker. Similar automatic control of the non-audio stimulation may be used, although the gain/depth control may be different for different modalities.

The switch in stimulation type (e.g., from sedative to stimulative) in block 706 may be done by an operator (e.g., the physician), may be based on time, may be based on sensors (e.g., EKG, pulse-ox, breathing rate), and/or triggered by connection to external environment (e.g., location inside the hospital, movement between speaker arrays, etc.). In an example, accelerometer data and/or EEG readings from one or more devices may detect a patient's return to consciousness and the modulation depth and gain of a piece of audio stimulation, or even the type of audio stimulation (e.g., from highly stimulating to more pleasant) may be changed. For example, audio stimulation with high gain/depth may be played when a patient is unconscious. Upon determining that the patient is about to regain consciousness, the audio stimulation may be switched to music that is very low gain/depth and is therefore pleasant, and it may ramp up from there to kickstart cognition.

IV. Example Clinical Study

Using sensory neuromodulation for recovery from anesthesia is being studied in an ongoing clinical study (registered at clinicaltrials.gov, ID NCT05291832) entitled, “A Randomized, Double-Blind, Placebo-Controlled Study to Explore Perioperative Functional Audio for Anxiety and Cognitive Recovery from Propofol Anaesthesia in Patients Undergoing Endoscopic Procedures,” and incorporated in U.S. Patent. App. No. 63/268,168, both of which are incorporated by reference herein in their entirety. The study includes a double-blinded randomized controlled trial with 220 patients undergoing elective colonoscopy or endoscopy procedures. The patients are assigned at random to hear either rhythmic auditory stimulation (music) or an active control placebo using spectrally-matched noise (i.e., sound that produces the same levels of activity at the cochlea but not expected to drive neural entrainment). Bone-conduction headphones are used by the patients for playback of the music (or matched noise). The music (or matched noise) is first administered in pre-operation waiting and consists of sedative music (or matched noise) until the propofol administration ceases, at which time the sedative music (or matched noise) will be switched to stimulative music (or matched noise).

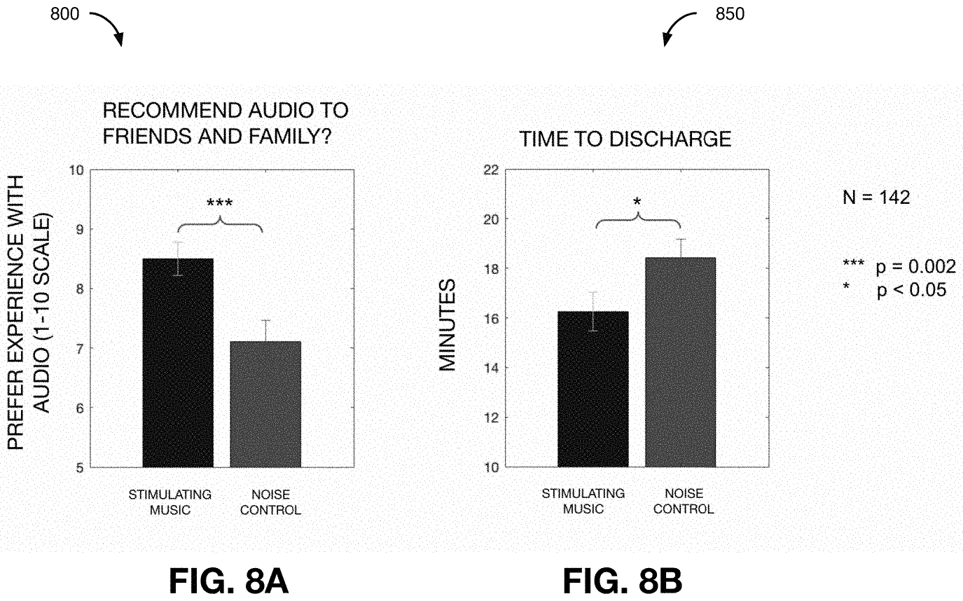

FIGS. 8A and 8B show preliminary results from the clinical study evaluating benefits of using stimulative music to aid recovery during the emergence from anesthesia. As part of the clinical study, participants are provided a survey to evaluate their recovery experience. FIG. 8A is a plot 800 showing the patient's willingness to recommend the audio they received to family and friends if undergoing the same procedure. On the y-axis of the plot 800, 10 represents the highest willingness to recommend audio, 5 represents no difference from the standard procedure without audio. As can be seen by the plot 800, patients who were administered stimulative music to recover from anesthesia were more likely to recommend the procedure with stimulative music over matched noise to their friends and family, and were much more likely to recommend the music over no audio (standard procedure). Statistical analysis with a t-test on these data showed that the results are highly statistically significant (with a 0.2% probability of having occurred by chance).

FIG. 8B is a plot 850 showing the average time to discharge a patient once they are in recovery (i.e., the time spent in postoperative care). As can be seen by plot 850, patients who were administered stimulative music to recover from anesthesia spent on average ˜13% less time in recovery than those that received matched noise. Statistical analysis with a t-test on these data showed a statistically significant difference (with a <5% probability of having occurred by chance). This result is practically of great importance as recovery time is often one of the biggest limiting factors on the rate of elective surgery at a facility since protocols often require an empty recovery bed prior to initiating a procedure.

Additional examples of the presently described method and device embodiments are suggested according to the structures and techniques described herein. Other non-limiting examples may be configured to operate separately or may be combined in any permutation or combination with any one or more of the other examples provided above or throughout the present disclosure.

It will be appreciated by those skilled in the art that the present disclosure may be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The terms “including” and “comprising” should be interpreted as meaning “including, but not limited to.” If not already set forth explicitly in the claims, the term “a” should be interpreted as “at least one” and the terms “the, said, etc.” should be interpreted as “the at least one, said at least one, etc.”

The present disclosure is described with reference to block diagrams and operational illustrations of methods and devices. It is understood that each block of the block diagrams or operational illustrations, and combinations of blocks in the block diagrams or operational illustrations, may be implemented by means of analog or digital hardware and computer program instructions. These computer program instructions may be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks may occur out of the order noted in the operational illustrations. For example, two blocks shown in succession may be executed substantially concurrently or the blocks may sometimes be executed in the reverse order, depending upon the functionality/acts involved.

For the purposes of this disclosure a non-transitory computer readable medium (or computer-readable storage medium/media) stores computer data, which data may include computer program code (or computer-executable instructions) that is executable by a computer, in machine readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, CD-ROM, DVD, or other optical storage, cloud storage, magnetic cassettes, magnetic tape, magnetic disk storage or other magnetic storage devices, or any other physical or material medium which may be used to tangibly store the desired information or data or instructions and which may be accessed by a computer or processor.

For the purposes of this disclosure the term “server” should be understood to refer to a service point which provides processing, database, and communication facilities. By way of example, and not limitation, the term “server” may refer to a single, physical processor with associated communications and data storage and database facilities, or it may refer to a networked or clustered complex of processors and associated network and storage devices, as well as operating software and one or more database systems and application software that support the services provided by the server. Cloud servers are examples.

For the purposes of this disclosure, a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router (WR) mesh, or 2^{nd}, 3^{rd}, 4^{th}, or 5^{th} generation (2G, 3G, 4G or 5G) cellular technology, Bluetooth, 802.11b/g/n, or the like. Network access technologies may enable wide area coverage for devices, such as client devices with varying degrees of mobility, for example. In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

It is the Applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

What is claimed is:

1. A method comprising:
receiving, by a processing device, an audio signal from an audio source;
receiving, by the processing device, a desired mental state;
identifying, by the processing device, an element of the audio signal that corresponds to a modulation characteristic of the desired mental state;
determining, by the processing device, an envelope from the element;
generating, by the processing device, one or more non-audio signals based on at least a rate and phase of the envelope; and
transmitting, by the processing device, the one or more non-audio signals to one or more non-audio output devices to generate one or more non-audio outputs.

2. The method of claim 1, wherein the modulation characteristic comprises one or more of a modulation rate, phase, depth, or waveform shape.

3. The method of claim 1, wherein the element comprises one or more of instruments, tempo, root mean square energy, loudness, event density, spectrum, temporal envelope, cepstrum, chromagram, flux, autocorrelation, amplitude modulation spectrum, spectral modulation spectrum, attack and decay, roughness, harmonicity, or sparseness.

4. The method of claim 1, wherein the generating comprises one or more of: ignoring amplitude differences of the element, altering a waveform shape of the modulation characteristic, and using a sub-band of the audio signal that is different than a sub-band of the envelope.

5. The method of claim 1, further comprising:
transmitting, by the processing device, the audio signal to one or more audio outputs to generate one or more audio outputs; and
coordinating, by the processing device, a relative timing of the more or more audio outputs and the one or more non-audio outputs.

6. The method of claim 5, wherein the coordinating is based on one or more predetermined models/rules.

7. The method of claim 6, wherein the coordinating is dynamically based on one or more sensors and comprises:
receiving, by the processing device, a sensor-input value from the one or more sensors;
determining, by the processing device, from a mapping of sensor-input values to stimulation parameters, revised stimulation parameters determined by the sensor-input value; and
modifying the generating the one or more non-audio signals based on the revised stimulation parameters.

8. The method of claim 7, wherein the one or more sensors comprise one or more of an accelerometer, a microphone, a camera, or a physiological sensor that measures heart rate, blood pressure, body temperature, electroencephalogram (EEG), magnetoencephalogram (MEG), Near infrared (fNIRS), or bodily fluid.

9. A device comprising a processor operative coupled to a memory, the memory configured to store instructions that, when executed by the processor, cause the processor to:
receive an audio signal from an audio source;
receive a desired mental state;
identify an element of the audio signal that correspond to a modulation characteristic of the desired mental state;
determine an envelope from the element;
generate one or more non-audio signals based on at least a rate and phase of the envelope; and
transmit the one or more non-audio signals to one or more non-audio output devices to generate one or more non-audio outputs.

10. The device of claim 9, wherein the modulation characteristic comprises one or more of a modulation rate, phase, depth, or waveform shape.

11. The device of claim 9, wherein the element comprises one or more of instruments, tempo, root mean square energy, loudness, event density, spectrum, temporal envelope, cepstrum, chromagram, flux, autocorrelation, amplitude modulation spectrum, spectral modulation spectrum, attack and decay, roughness, harmonicity, or sparseness.

12. The device of claim 9, wherein the generating comprises one or more of: ignoring amplitude differences of the element, altering a waveform shape of the modulation characteristic, and using a sub-band of the audio signal that is different than a sub-band of the envelope.

13. The device of claim 9, wherein the instructions, when executed by the processor, further cause the processor to:
transmit the audio signal to one or more audio output devices to generate one or more audio outputs; and
coordinate a relative timing of the more or more audio outputs and the one or more non-audio outputs.

14. The device of claim 13, wherein the coordinating is based on one or more predetermined models/rules.

15. The device of claim 13, wherein the coordinating is dynamically based on one or more sensors and the instructions, when executed by the processor, further cause the processor to:
receive a sensor-input value from the one or more sensors;
determine, from a mapping of sensor-input values to stimulation parameters, a revised modulation characteristic that corresponds to the sensor-input value; and
modify the generating the one or more non-audio signals based on the revised modulation characteristic.

16. The device of claim 15, wherein the one or more sensors comprise one or more of an accelerometer, a microphone, a camera, or a physiological sensor that measures heart rate, blood pressure, body temperature, electroencephalogram (EEG), magnetoencephalogram (MEG), Near infrared (fNIRS), or bodily fluid.

17. A method of using neuromodulation to improve patient experience before, during, and after anesthesia, the method comprising:
administering rhythmic sensory stimulation to have a sedative effect prior to administration of the anesthesia; and
modifying the rhythmic sensory stimulation to have a stimulative effect after administration of the anesthesia has concluded,
wherein the rhythmic stimulation comprises an audio output generated by an audio device and a non-audio output generated by a non-audio device.

18. The method of claim 17, wherein the audio devices comprise one or more of bone-conduction headphones, pass-through headphones, and nearfield speakers and the non-audio devices comprise one or more wearables, a connected vibrating bed, and lights.

19. The method of claim 17, wherein the modifying occurs while the patient is unconscious and is performed by one or more of a manual selection by a caregiver or an automatic selection based on one or more sensors.

20. The method of claim 17, further comprising:
adjusting one or more characteristics of the rhythmic sensory stimulation via one or more of manual input by one or more of the patient and a caregiver and automatic input based on one or more sensors, wherein the one or more characteristics comprise gain and modulation depth.
